(19) United States
(12) Patent Application Publication
(10) Pub. No.: US 2019/0343470 A1
(43) Pub. Date: Nov. 14, 2019

(54) PATIENT COUCH AND X-RAY SYSTEM WITH SUCH A PATIENT COUCH

(71) Applicant: Siemens Healthcare GmbH, Erlangen (DE)

(72) Inventor: THOMAS DIPPL, Pressath (DE)

(73) Assignee: Siemens Healthcare GmbH, Erlangen (DE)

(21) Appl. No.: 16/404,850

(22) Filed: May 7, 2019

(30) Foreign Application Priority Data
May 11, 2018 (DE) 102018207375.9

(51) Int. Cl. A61B 6/04; A61B 5/055

(52) U.S. Cl. CPC A61B 6/0407; A61B 5/704; A61B 5/0555

(57) ABSTRACT

A patient couch, in particular for radiography and/or fluoroscopy, includes an adjustable patient support having longitudinal and face sides. According to an embodiment, the patient support is supported in a rotationally and vertically adjustable manner on a least one vertical column at least by way of one of its longitudinal sides. An x-ray system with a patient couch embodied in this manner is also proposed.

### PRIORITY STATEMENT

The present application hereby claims priority under 35 U.S.C. § 119 to German patent application number DE 102018207375.9 filed May 11, 2018, the entire contents of which are hereby incorporated herein by reference.

### FIELD

At least one embodiment of the invention relates to a patient couch, in particular for radiography and/or fluoroscopy, with an adjustably supported patient support having longitudinal and face sides. The invention further relates to an x-ray system with at least one x-ray emitter and one such patient couch.

### BACKGROUND

Patient couches, in particular for radiography or fluoroscopy, with adjustable patient supports having longitudinal and face sides are known sufficiently from the prior art. The patient support serves for instance to suitably position an examination-relevant body region of a patient in particular between an x-ray emitter and an x-ray detector for image detection.

In particular, with the continuous observation of dynamic processes using fluoroscopy, it may be necessary for the patient to assume a specific posture during the image detection process. A flexible positioning of the patient support is therefore desirable.

U.S. Pat. No. 5,870,450 discloses a patient couch with a patient support, which is rotatably supported laterally on a base unit by way of its longitudinal side. The patient support is moreover adjustable, so that contact with the floor can be avoided when the patient support is pivoted. The patient support is fastened to the base unit in a central region of the couch plane.

### SUMMARY

At least one embodiment of the present invention specifies a space-saving concept of a patient support, in particular for an x-ray system.

At least one embodiment of the invention is directed to a patient couch.

Advantageous embodiments of the invention form the subject matter of the claims.

At least one embodiment of the present invention is directed to a patient couch, in particular for radiography and/or fluoroscopy, comprising an adjustable patient support having longitudinal and face sides. The patient support is supported in a rotationally and vertically adjustable manner on at least one vertical column at least by way of one of its longitudinal sides.

At least one embodiment of the present invention further relates to an x-ray system, in particular imaging radiography and/or fluoroscopy x-ray system, with at least one x-ray emitter and the previously described patient couch.

### BRIEF DESCRIPTION OF THE DRAWINGS

For a further description of the invention, reference is made to the example embodiments shown in the figures of the drawing, in which, shown schematically:

FIG. 1: shows an x-ray system, comprising a patient couch with an adjustable patient support in a first positioning;

FIG. 2: shows an x-ray system, comprising a patient couch with an adjustable patient support in a second positioning;

FIG. 3: shows an x-ray system, comprising a patient couch with an adjustable patient support in a third positioning;

Parts which correspond to one another are provided with the same references in all the figures.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection.

Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising, ” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the present invention is directed to a patient couch, in particular for radiography and/or fluoroscopy, comprising an adjustable patient support having longitudinal and face sides. The patient support is supported in a rotationally and vertically adjustable manner on at least one vertical column at least by way of one of its longitudinal sides.

The lateral suspension of the patient support on one or more vertical columns arranged laterally with respect to the patient support allows for adjustment by way of a further angle and positional range. Here it is possible in particular to dispense with the use of telescopic components or telescopic legs.

According to at least one embodiment of the invention, the patient support is connected at at least one bearing point with the at least one vertical column, which is arranged offset with respect to the couch plane in the direction of a face-side, in particular head-side, end of the patient support. In this sense, the bearing point is positioned eccentrically with respect to the couch plane. In this way, considerably less space is used by a rotational or tilting movement with a combined height adjustment, which brings the patient support in particular from one horizontal position into a vertical position, than would be necessary for instance with a centrical suspension of the patient support.

The vertical column or columns which are used to suspend the patient support are arranged in particular laterally offset with respect to a couch plane of the patient support.

In one embodiment, the patient support is supported rotatably on the at least one vertical column about an axis of rotation running parallel to the face side. The patient support has a smaller lateral extent, particularly along its face side, than along its longitudinal side. In this sense the patient support is supported rotatably about a transverse axis.

A positioning in which the couch plane of the patient support runs in a horizontal plane is in particular understood to mean a horizontal position of the patient support. A positioning in which the couch plane of the patient support runs in a vertical plane is in particular understood to mean a vertical position of the patient support.

In one embodiment, the at least one vertical column has a vertical guide for adjusting the height of the patient support. With a corresponding design of the vertical guide, very low step heights can be realized with this concept.

In one embodiment, the patient support is supported rotatably in the vertical guide.

In one embodiment an x-ray detector is integrated in the patient support. Patient supports embodied in such a way are embodied in particular for use in a fluoroscopy or conventional radiography x-ray system.

In one embodiment, the x-ray detector is supported movably both longitudinally and transversally with respect to a couch plane of the patient support, in particular in order to cover as large a detection range as possible.

In one embodiment, the patient support is supported rotatably by at least 90° with respect to the at least one vertical column such that the patient support can be adjusted at least between a horizontal and a vertical position. In this way, the patient in particular can be moved from a horizontal position into a vertical position. This allows x-ray images of an examination region to be recorded in lines of sight which run at right angles to one another.

In one embodiment, the patient support is supported in a rotationally and vertically adjustable manner by way of two opposing longitudinal sides on vertical columns arranged opposite one another. The two vertical columns are arranged in particular laterally offset with respect to the couch plane of the patient support and opposite one another.

In one alternative embodiment hereto, the patient support is supported in a rotationally and vertically adjustable manner on a vertical column by way of one of its longitudinal sides. In other words, the patient support is only connected to one of its longitudinal sides with a single vertical column arranged laterally offset with respect to the couch plane.

In one embodiment, the patient couch has at least one optical sensor, in particular 3D camera, for detecting the position of at least the patient support and/or a patient. The optical sensor is connected to a control unit, which is embodied to control at least one drive which adjusts the position and/or alignment of the patient support in accordance with the positions detected. The control unit is therefore embodied to position or align, automatically or autonomously, the patient support in accordance with the data characterizing the position of the patient and/or of the patient support.

Alternatively, the optical sensor, in particular the 3D camera is set up separately from the patient couch in an examination room. In this case, the optical sensor is actively connected to the control unit and the drive unit in such a way that the position and/or alignment of the patient support can be controlled in accordance with the detected positions. To this end, the optical sensor can be embodied for instance to communicate with the control unit in a wireless or wired manner.

At least one embodiment of the present invention further relates to an x-ray system, in particular imaging radiography and/or fluoroscopy x-ray system, with at least one x-ray emitter and the previously described patient couch.

In one embodiment, the at least one x-ray emitter is movably supported by a ceiling or base stand, for instance, and at least one optical sensor, in particular a 3D camera, is provided for detecting the position of at least the patient support and/or a patient and/or the x-ray emitter. The position detection preferably involves detecting the position and alignment of the object to be detected in the space. The optical sensor is connected to a control unit which is embodied to control at least one drive adjusting the position and/or alignment of the patient support and/or of the x-ray emitter in accordance with the detected positions. An automatic or autonomous adjustment of the relative distances and alignment of the patient support and the x-ray emitter relative to one another preferably takes place. As a function of the medical use, x-ray image recordings at distances of up to a few meters, for instance at a distance of 3 m, are in particular enabled.

The control unit is integrated for instance in the at least one vertical column of the patient couch.

Alternatively or in addition, components of the system electronics and/or an image system computer embodied in particular to evaluate detected x-ray images and comprising processors for instance and/or a generator are integrated in the at least one vertical column of the patient couch.

In one embodiment, an x-ray detector which is supported in a longitudinally and/or transversally moveable manner with respect to a couch plane is integrated in the patient support. The control unit is embodied to control at least one further drive adjusting the position of the x-ray detector in accordance with the detected positions. An automatic or autonomous adjustment of the relative position and/or alignment of the patient support, of the x-ray emitter and of the x-ray detector preferably takes place in particular such that the patient does not have to be relocated or moved during the examination.

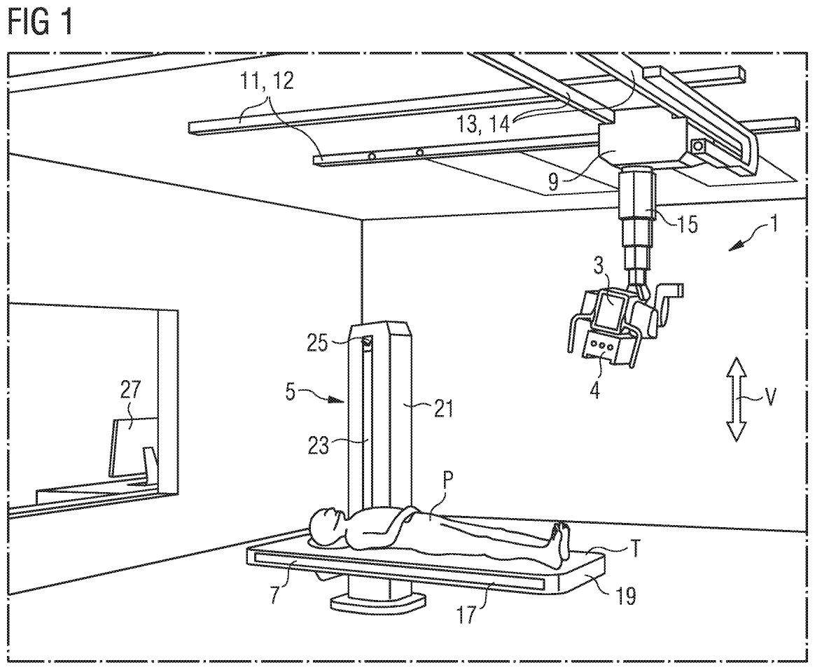

FIGS. 1 to 3 show an x-ray system 1 with a movably supported x-ray emitter 3, a screen unit 4 and a patient couch 5, which has a height-adjustable and rotatable patient support 7. An x-ray detector for detecting x-ray radiation is integrated within the patient support 7 in a manner not shown. The x-ray detector is arranged in a longitudinally and/or transversally movable manner within the patient support 7 so that this can be suitably positioned with respect to the x-ray emitter, in particular such that a central beam emitted by the x-ray emitter strikes the x-ray detector centrally.

In the example embodiment shown the x-ray emitter 3 is supported movably, in particular pivotably, on a ceiling stand 9, so that this can be positioned and aligned almost arbitrarily in the space. For translation in a horizontally running plane, the ceiling stand 9 is supported displaceably on guides 11, 13 with guide rails 12, 14 running at right angles to one another. For translation of the x-ray emitter 3 in the vertical direction V, the ceiling stand 9 is embodied with a telescopic arm 15, to which it is attached at the end.

For the purpose of supporting a patient P, the patient support 7 defines a couch plane T, which can be aligned in various angular positions with respect to the gravity field and can be adjusted vertically along the vertical direction V. The support range of the patient support 7 for the patient P has a substantially rectangular design with longitudinal sides 17 and face sides 19 running at right angles thereto. The longitudinal sides 17 have a larger lateral extent than the face sides 19.

The patient support 7 of the example embodiment shown is only supported in a rotationally and vertically adjustable manner on one side on a vertical column 21 by way of one of the longitudinal sides 19. To this end, the patient support 7 is supported in a rotatably and displaceable manner in particular in a vertical guide 23, which extends along the vertical column 21 in the vertical direction V. The bearing point of the patient support is arranged eccentrically with respect to the couch plane T, in particular offset in the direction of the head-side face side 19. A suspension of the patient support 7 of this type allows the patient support 7 to be pivoted at the same time as adjusted vertically, as illustrated schematically in FIGS. 1 to 3, by way of a further, in particular continuous angle range, wherein the space requirement needed for the movement is minimal. In this way the axis of rotation, about which the patient support 7 can be rotated, runs parallel to the face sides 19.

FIG. 1 shows the patient support 7 in the horizontal position. The couch plane T extends in a horizontal plane at right angles to the gravity field.

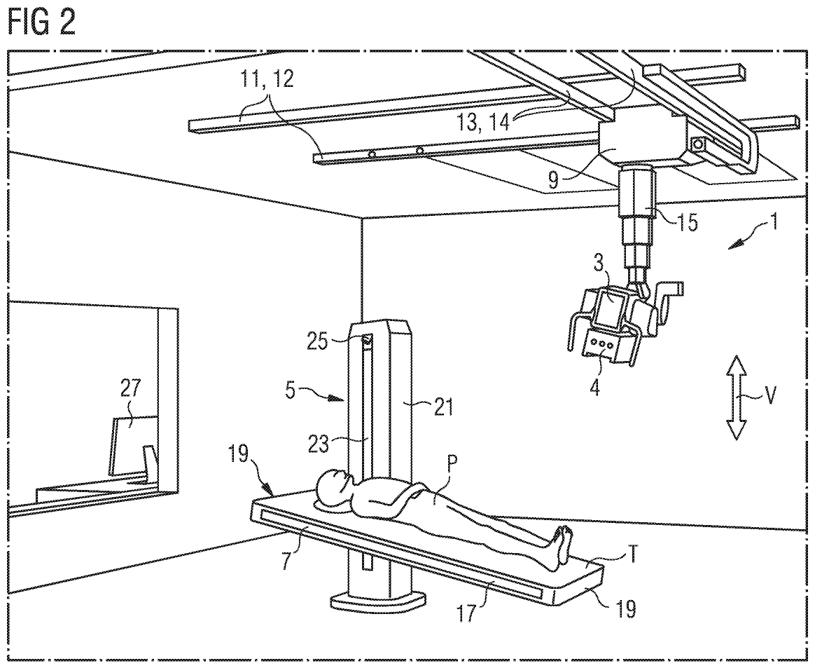

In FIG. 2 the patient support 7 is pivoted about approximately 20° with respect to the horizontal position.

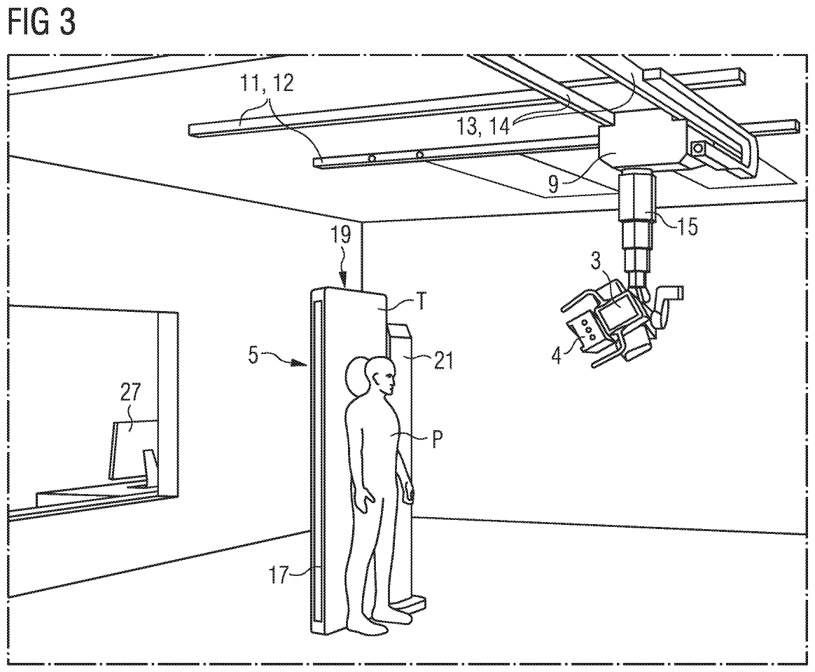

In FIG. 3 the patient support 7 is pivoted about 90° with respect to the horizontal position. The couch plane T extends in the vertical direction, in other words in a plane parallel to the vertical direction V.

An optical sensor 25, which is embodied as a 3D camera, is attached to the top end of the vertical column 21. The optical sensor 25 detects the surroundings of the x-ray system 1, in particular the position and alignment of the patient support 7, of the patient P and of the x-ray emitter 3. A control unit is integrated in the vertical column and is embodied to control actuators, in particular drives, which adjust the position or alignment of the detected components, in particular the x-ray emitter 3, the patient support 7 and the x-ray detector, in accordance with the detected positions or position data. In this way, an x-ray system 1 is specified, which is embodied to autonomously align or position imaging components, such as in particular the x-ray emitter 3 and the x-ray detector, and the patient support 7 for the image detection. The detected x-ray images can be displayed for instance by way of a display device 27, for instance a monitor. With fluoroscopy x-ray systems 1, a continuous display of a physiological process is in particular provided by way of the display device 27.

Although the invention has been illustrated and described in detail with respect to the preferred example embodiments, the invention is not restricted hereto. Other variations and combinations can be derived herefrom by the person skilled in the art, without deviating from the essential idea of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A patient couch, comprising:
an patient support including longitudinal sides and face sides, the patient support being adjustable, supported in a rotationally and vertically adjustable manner on at least one vertical column, at least by way of one of the longitudinal sides, and the patient support being connected at at least one bearing point with the at least one vertical column, arranged offset with respect to a couch plane in a direction of one face-side of the face-sides.

2. The patient couch of claim 1, wherein the patient support is supported rotatably on the at least one vertical column about an axis of rotation running parallel to the one face side.

3. The patient couch of claim 1, wherein the at least one vertical column includes a vertical guide for height adjustment of the patient support.

4. The patient support of claim 3, wherein the patient support is supported rotatably in the vertical guide.

5. The patient couch of claim 1, wherein an x-ray detector is integrated in the patient support.

6. The patient couch of claim 5, wherein the x-ray detector is supported in at least one of a longitudinally and transversally movable manner with respect to a couch plane of the patient support.

7. The patient couch of claim 1, wherein the patient support is supported rotatably about at least 90° with respect to the at least one vertical column such that the patient support is adjustable at least between a horizontal and a vertical position.

8. The patient couch of claim 1, wherein the patient support is supported in a rotationally and vertically adjustable manner on oppositely arranged vertical columns by way of two opposing longitudinal sides.

9. The patient couch of claim 1, wherein the patient support is supported on one side in a rotationally and vertically adjustable manner on a vertical column by way of one of the longitudinal sides.

10. The patient couch of claim 1, further comprising:
at least one optical sensor to detect a position of at least one of the patient support and a patient, the optical sensor being connected with a control unit, embodied to control at least one of drive adjusting the position and alignment of the patient support in accordance with the position detected.

11. An x-ray system, comprising:
at least one x-ray emitter; and
the patient couch of claim 1.

12. The x-ray system of claim 11, wherein the at least one x-ray emitter is supported movably, and wherein the x-ray system further comprises: at least one optical sensor to detect a position of at least one of the patient support, a patient and the x-ray emitter, wherein the optical sensor is connected to a control unit, embodied to control at least one of drive adjusting the position and alignment of at least one of the patient support and the x-ray emitter, in accordance with the position detected.

13. The x-ray system of claim 12, further comprising:
an x-ray detector, supported in at least one of a longitudinally and transversally movable manner with respect to a couch plane, integrated in the patient support, and wherein the control unit is embodied to control at least one further drive to adjust the position of the x-ray detector in accordance with the position detected.

14. The patient couch of claim 1, wherein the patient couch is for at least one of radiography and fluoroscopy.

15. An x-ray system for radiography or fluoroscopy, comprising:
at least one x-ray emitter; and
the patient couch of claim 1.

16. The patient couch of claim 1, wherein the patient support is connected at at least one bearing point with the at least one vertical column, arranged offset with respect to a couch plane in a direction of a head-side end of the patient support.

17. The patient couch of claim 2, wherein the at least one vertical column includes a vertical guide for height adjustment of the patient support.

18. The patient couch of claim 2, wherein an x-ray detector is integrated in the patient support.

19. The patient couch of claim 10, wherein the at least one optical sensor is a 3D camera.

20. The x-ray system of claim 12, wherein the at least one optical sensor is a 3D camera.
